(19) United States
(12) Patent Application Publication
(10) Pub. No.: US 2014/0017178 A1
(43) Pub. Date: Jan. 16, 2014

(54) Blue Curls "Pirzada" and Related Compositions and Methods

(71) Applicant: Syed Zebe Pirzada, San Marino, CA (US)

(72) Inventor: Syed Zebe Pirzada, San Marino, CA (US)

(21) Appl. No.: 13/997,113

(22) PCT Filed: Dec. 21, 2011
(86) PCT No.: PCT/US11/66506

§ 371 Date: Sep. 13, 2013

Related U.S. Application Data
(60) Provisional application No. 61/426,960, filed on Dec. 23, 2010.

(51) Int. Cl. C07C 35/18; A61K 31/045; A61K 36/53

(52) U.S. Cl. CPC C07C 35/18; A61K 36/53; A61K 31/045
USPC 424/49; 568/825; 424/745

(57) ABSTRACT

The present disclosure embrace Blue Curls (Trichostema lanatum) var. Pirzada and compositions and methods there from. Specifically, the applications provides methods and compositions relating to Blue Curls Pirzada essential oil and floral water.

### TECHNICAL FIELD

The present disclosure relates to Trichostema lanatum var. “Pirzada” (“Blue Curls ‘Pirzada’”) and compositions and methods there from.

### INTRODUCTION

Trichostema lanatum (common name “Blue Curls”) is a small evergreen shrub native to southern California and is a member of the Mint (Lamiaceae) family. Blue Curls grows to about two feet, and has many branches with narrow, pointy, green leaves. Blue Curls flowers arise in dense clusters, and the flowers are blue or purple in color and are covered with a dense coat of white wooly hair.

Despite its attractive appearance, Blue Curls is not a common garden plant. Blue Curls' lack of popularity may be attributed to the fact that while the plant produces attractive flowers for about five years, subsequent years result in a “lion tailed” appearance wherein new growth occurs at the tips while lower branches become bare. Additionally, Blue Curls provides no known economic product, though folklore suggests that early Californians boiled Blue Curls leaves and flowers to make tea, as well as frying the flowers in olive oil to make an ointment for pain and ulcer care. Parsons, M. E., 1907. The Wildflowers of California. Cunningham, Curtis & Welch. Republished in 1966 by Dover Publications, New York. Despite these anecdotal references, Blue Curls' natural chemistry remains largely uninvestigated, if not unpublished. Blue Curls, for example, does not appear in the natural product or chemical literature, and is absent in modern pharmacopeias.

### SUMMARY

In one aspect, there is provided a method for obtaining terpinen-4-ol, comprising (a) growing a Blue Curls ‘Pirzada’ plant; and (b) isolating terpinen-4-ol from Blue Curls ‘Pirzada.’ In one embodiment, the terpinen-4-ol is isolated from gland-tipped epidermal trichomes.

In another aspect, there is provided an essential oil isolated from Blue Curls ‘Pirzada,’ wherein said oil comprises at least about 60% terpinen-4-ol.

In another aspect, provided herein is a composition comprising an essential oil isolated from Blue Curls ‘Pirzada,’ wherein said oil comprises at least about 60% terpinen-4-ol. In one embodiment, the composition is a pharmaceutical, nutraceutical, cosmetic, lotion, toothpaste, or fragrance.

In another aspect, there is provided a method for treating a burn, comprising (a) isolating terpinen-4-ol from Blue Curls ‘Pirzada’; and (b) applying said terpinen-4-ol on burn tissue.

In another aspect, there is provided a method for treating a microbial infection, comprising (a) isolating terpinen-4-ol from Blue Curls ‘Pirzada’; and (b) applying said terpinen-4-ol on a surface comprising microbial infestation. In one embodiment, the surface is skin.

In another aspect, there is provided a Blue Curls ‘Pirzada’ plant, as deposited with the American Type Culture Collection as deposit number XXXX.

In another aspect, there is provided a method for making a pharmaceutical, comprising (a) growing a Blue Curls ‘Pirzada’ plant; (b) isolating terpinen-4-ol from Blue Curls ‘Pirzada’; and (c) combining said terpinen-4-ol into with a carrier.

In another aspect, there is provided a method for increasing terpinen-4-ol content in a plant, comprising crossing a plant with a Blue Curls ‘Pirzada’ plant, wherein Blue Curls ‘Pirzada’ plant is either the pollen or egg donor.

In another aspect, there is provided an isolated essential oil comprising at least 60% terpinen-4-ol.

In another aspect, there is provided a floral water isolated from Blue Curls ‘Pirzada,’ wherein said floral water comprises at least about 60% terpinen-4-ol.

In another aspect, provided herein is a composition comprising floral water isolated from Blue Curls ‘Pirzada,’ wherein said floral water comprises at least about 60% terpinen-4-ol.

### DETAILED DESCRIPTION

The present inventor selected a botanical Blue Curls in California that produces an essential oil containing very high levels of terpinen-4-ol. This Blue Curls variety, now named ‘Pirzada’ after the inventor's surname, is a fast-growing annual that produces about 60% terpinen-4-ol, which translates into about 600 ml of terpinen-4-ol per liter of Blue Curls essential oil.

Terpinen-4-ol is a trace compound found in many essential oils. Presently, the main source of terpinen-4-ol is the Australian tea tree (Melaleuca alt.), which provides about 38% terpinen-4-ol, which translates into about 380 ml of terpinen-4-ol per liter of tea tree essential oil. Terpinen-4-ol can be used in a variety of compositions and applications, and is valued throughout the world for its antimicrobial properties. Because Blue Curls ‘Pirzada’ produces nearly 1000 seeds per plant and has no known plant competitor, Blue Curls ‘Pirzada’ provides a rapid and inexpensive source of terpinen-4-ol.

Furthermore, and as described in greater detail below, the present inventor discovered that Blue Curls ‘Pirzada’ essential oil provides several advantages over Australian tea tree oil. In addition to the fact that Blue Curls ‘Pirzada’ essential oil is higher in terpinen-4-ol than Australian tea tree oil, Blue Curls ‘Pirzada’ essential oil lacks camphoraceous compounds that can irritate skin. Blue Curls ‘Pirzada’ essential oil comprises many aromatic esters, which can yield a fragrant product itself, or integrate into another product. Likewise, Blue Curls ‘Pirzada’ flowers have a very fragrant aroma and thus the flower material can be used, for example, in a perfume or cologne formulation. Conversely, Australian tea tree has an objectionable odor and has not found use in the fragrance industry. Furthermore, Blue Curls ‘Pirzada’ essential oil comprises terpinen-4-ol acetate (12.5%), which tea tree oil does not have.

All technical terms used herein are terms commonly used in biochemistry, chemistry, plant biology, plant physiology, botany, plant taxonomy, and agriculture, and can be understood by one of ordinary skill in the art. Those technical terms can be found in: Plant Physiology, 2^{nd} Edition, Taiz and Zeiger (Sinauer Associates, 1998); Introduction to Plant Physiology, William Hopkins (John Wiley & Sons, Inc., 1995); Anatomy of Seed Plants, Katherine Esau ((John Wiley & Sons, Inc., 1960); Plant Systematics: A Phylogenetic Approach, Campbell, Kellogg, Stevens, Donoghue & Judd (Sinauer Associates, 1998); Economic Botany: Plants in Our World, 2^{nd} Edition, Simpson and Ogorzaly (McGraw-Hill, Inc., 1995), and the like.

“Blue Curls” (Trichostema lanatum) is a small evergreen shrub native to southern California and is a member of the Mint (Lamiaceae) family. Disclosed herein is a new Blue Curls variety, “Pirzada,” that has an essential oil with unique characteristics, including but not limited to high terpinene-4-ol content (about 60% (w/v)), high terpenoid and sesquiterpenoid floral esters content, and negligible camphoraceous compounds.

“Essential oil” refers to a complex mixture of volatile compounds produced by a small percentage of botanical species referred to as “aromatic plants.” Essential oils can be used in a variety of applications, including but not limited to internal, external, or aromatic medicinal preparations; culinary preparations; scent and fragrances; and industrial chemical precursors.

Terpinene-4-ol is a terpene and is believed the primary active ingredient of tea tree oil. Tea tree oil is valued for it antimicrobial activity, as well as finding use in reducing pain and inflammation, acne, warts, boils, and inhibits infections associated with first and second-degree burns, while preventing scarring. Tea tree oil has been used for treating fungal infection, scalp irritation, gingivitis, and a variety of skin conditions.

Floral water or hydrosol refers to the aqueous portion of the distillate obtained when an aromatic botanical is steam distilled. Generally, during the distillation of economically-important aromatic plants, the floral water is discarded as a waste product. Certain exceptions include the floral waters of rose and lavender, both of which are used either independently or combined into other products. Floral waters are gaining popularity as bases for hand creams, cosmetics, splashes, and bath preparations.

### A. Plant Material

“Blue Curls” refers to a plant of the Lamiaceae family, specifically Trichostema lanatum variety ‘Pirzada’ selected in Bradley, Calif. In 1994, inventor Zebe Pirzada and his family found a plant on their land, that was oily to the touch. After analyzing the plant oil by gas chromatography, it was determined that the oil comprised terpinen-4-ol. Using bulk seed selection methodology, inventor Pirzada collected seeds from the analyzed plants and planted the seeds to generate progeny plants. Seeds from the most vigorous and tallest progeny plants were bulk collected and planted. This process was repeated iteratively for over four years, wherein each year, the seed from selected plants were bulked for the next generation and planted. Following four years of successive bulk selection, inventor Pirzada generated thousands of seeds, wherein the resultant plants displayed high terpinene-4-ol content (about 60% (w/v)), high terpenoid and sesquiterpenoid floral esters content, and negligible camphoraceous compounds.

Exemplary Blue Curls ‘Pirzada’ seeds were deposited with the American Type Culture Collection (Manassas, Va.) on XXXX and accessible under accession number XXXX. Of course, and as known in the art, Blue Curls ‘Pirzada’ can be propagated by any suitable plant breeding method, including seed such as those deposited with the ATCC. Additionally, Blue Curls ‘Pirzada’ can be produced by crossing a plant with a Blue Curls ‘Pirzada’ plant, wherein the Blue Curls ‘Pirzada’ plant is either the pollen or egg donor.

“Blue Curls” plants are cultivated by seeds in the early spring. For example, fields are cultivated by light grading, and then seeds are planted in the field close to the surface. Because “Blue Curls” prefers arid soil, generally, sufficient water is provided by spring rain, though additional water can be provided by irrigation if needed. “Blue Curls” prefers strong sunlight and heat.

“Blue Curls” is ready for harvest in the fall, generally around the end of September or early October in California. Blue Curls essential oil is produced in the epidermis, specifically in gland-tipped epidermal trichomes (plant hairs) that are distributed on the surfaces of the leaves (both adaxial and abaxial sides,) stem, and the abaxial face of the calyx.

### B. Isolation of Blue Curls ‘Pirzada’ Essential Oil

This disclosure provides an essential oil extracted from Blue Curls ‘Pirzada.” While an essential oil can be isolated from a plant in any suitable manner, exemplary isolation or extraction methods include distillation, for example, steam distillation. For example, the essential oil can be extracted in a two stage distillation process (double distillation). During double distillation, the oil is first extracted by steam distillation (at a temperature of about 100 degrees C.) to remove most impurities.

For example and in no way limiting, Blue Curls essential oil can be isolated by steam distillation. In one steam distillation protocol, Blue curls essential oil passes through a condenser thereby facilitating extraction of the oil and the floral waters such that oil and the floral water remain in one component. In another steam distillation method, steam is injected into plant matter via a steam engine and the entire product goes through a cooling process into a still. Once in the still, pressure is injected which raises and separates the oil into another component where the oil is separated and removed. Floral waters can be removed and placed into a drum or other storage for bottling.

Thus, in one embodiment, following harvest of “Blue Curls” tissue, Blue Curls essential oil is extracted from the epidermis, specifically in gland-tipped epidermal trichomes that are distributed on the surfaces of the leaves (both adaxial and abaxial sides,) stem, and the abaxial face of the calyx via steam distillation.

### C. Compositions

A variety of compositions can be made using Blue Curls ‘Pirzada’ essential oil. For example and in no way limiting, pharmaceutical compositions can include oil extracted from Blue Curls. In one non-limiting example, an antimicrobial pharmaceutical can be obtained from Blue Curls plant oil extracts. As used herein, “antimicrobial activity” includes bactericidal, fungicidal, protozoanicidal, and/or any other activity that prevents microbial colonization and growth. Such pharmaceutical compositions are suitable for treating internal and external microbial infections in animals, including, humans and livestock, including but not limited to horses, cows, pigs, sheep, goats, rabbits, dogs, cats and poultry, including, but not limited to chickens, turkeys, ducks and pet birds. Similarly, Blue Curls extracts can be used for herbals and related nutraceuticals.

Likewise, Blue Curls extracts may find use in fragrance and/or other aromatic compositions. Generally, and as known in the art, fragrance results from a variety of components blended together, such as essential oils, extracts from woods, gums, flowers and other botanicals, resins, animal secretions, and synthetic aromatic materials. For example, a fragrance may use an essential oil from Blue Curls, such as the essential oil from Blue Curls ‘Pirzada.’ Exemplary fragrances and related aromatics, such as fabric fresheners, air fresheners, and other consumer fragrances, can be made from Blue Curls ‘Pirzada’ essential oil using methods known in the art, such as those described in standard texts known within the perfume industry, particularly: “Common Fragrance and Flavor Materials” by Bauer, Garbe and Surburg, VCH Publ., 3.sup.rd edition (2001), “Perfume and Flavor Materials”, Steffen Arctander (Montclair, N.J., 1969) published in two volumes, “Perfume and Flavor Materials of Natural Origin”, S. Arctander (Elizabeth, N.J., 1960).

Of course, and as known in the art, other consumer products can be made from the essential oil from Blue Curls ‘Pirzada’ including but not limited to mouthwash, dental floss, lip balm, salve, lotion, toothpaste, deodorant, antiperspirant, bandages, and a host of other cosmetic and health care products.

The following examples demonstrate non-limiting embodiments. However, those of ordinary skill in the art appreciate that many changes can be made and still obtain a like or similar result without departing from the spirit and scope of the disclosure.

### EXAMPLE 1

### Plant Material

Blue Curls ‘Pirzada’ was identified and selected in Bradley, Calif. In 1994, inventor Zebe Pirzada and his family found a plant on their land, that was oily to the touch. After analyzing the plant oil by gas chromatography, it was determined that the oil comprised terpinen-4-ol. Seeds from the plant were sown and resulted plants were selected for high terpinene-4-ol content (about 60% (w/v)), high terpenoid and sesquiterpenoid floral esters content, and negligible camphoraceous compounds.

Exemplary Blue Curls ‘Pirzada’ seeds were deposited with the American Type Culture Collection (Manassas, Va.) on XXXX and accessible under accession number XXXX.

Blue Curls ‘Pirzada’ plants are cultivated by seeds in the early spring. For example, fields are cultivated by light grading, and then seeds are planted in the field close to the surface. Because Blue Curls ‘Pirzada’ prefers arid soil, generally, sufficient water is provided by spring rain, though additional water can be provided by irrigation if needed. Blue Curls ‘Pirzada’ prefers strong sunlight and heat.

Blue Curls ‘Pirzada’ is ready for harvest in the fall, generally around the end of September or early October in California. As described below in Example 2, freshly harvested Blue Curls leaves are used for isolating essential oil, as the essential oil is produced in the epidermis, specifically in gland-tipped epidermal trichomes (plant hairs) that are distributed on the surfaces of the leaves (both adaxial and abaxial sides,) stem, and the abaxial face of the calyx.

### EXAMPLE 2

### Isolation of Blue Curls ‘Pirzada’ Essential Oils

Freshly harvested Blue Curls ‘Pirzada’ tissue is used for extracting essential oil from Blue Curls ‘Pirzada.’ While any tissue containing oil-producing may be used, the present inventor isolates Blue Curls ‘Pirzada’ essential oil from gland-tipped epidermal trichomes that are distributed on the surfaces of the leaves (both adaxial and abaxial sides,) stem, and the abaxial face of the calyx via steam distillation.

While an essential oil can be isolated from a plant in any suitable manner, exemplary isolation or extraction methods include distillation, for example, steam distillation. For example, the essential oil can be extracted in a two stage distillation process (double distillation). During double distillation, the oil is first extracted by steam distillation (at a temperature of about 100 degrees C.) to remove most impurities.

For example and in no way limiting, Blue Curls essential oil can be isolated by steam distillation. In one steam distillation protocol, Blue curls essential oil passes through a condenser thereby facilitating extraction of the oil and the floral waters such that oil and the floral water remain in one component. In another steam distillation method, steam is injected into plant matter via a steam engine and the entire product goes through a cooling process into a still. Once in the still, pressure is injected which raises and separates the oil into another component where the oil is separated and removed. Floral waters can be removed and placed into a drum or other storage for bottling.

### EXAMPLE 3

### Characterization of Essential Oils

As described in Example 2 above, freshly harvested Blue Curls ‘Pirzada’ tissue was used for extracting essential oil from Blue Curls ‘Pirzada.’ After extracting the essential oil via steam distillation and fractioning the resultant essential oil and floral water, the essential oil was characterized by gas chromatography and mass spectrometry (GC/MS). The results are shown in Table 1 below.

TABLE 1
Gas Chromatography Analysis of Blue Curls
‘Pirzada’ Essential Oil

| | | | Retention Time | Area |
|---|---|---|---|---|
| Peak No. | Peak Name | Result | (minutes) | (counts) |
| 1 | a-pinene | 1.0074 | 3.485 | 22934 |
| 2 | B-pinene | 0.0705 | 4.437 | 1605 |
| 3 | Sabinene | 0.0516 | 4.578 | 1175 |
| 4 | Myrcene | 0.1399 | 5.152 | 3185 |
| 5 | a-Phellandre | 1.0053 | 5.289 | 22887 |
| 6 | a-Terpinene | 3.5385 | 5.560 | 80559 |
| 7 | Limonene | 0.1044 | 5.935 | 2377 |
| 8 | B-Phel./Euca | 1.5686 | 6.160 | 37513 |
| 9 | g-Terpinene | 7.2541 | 7.012 | 165151 |
| 10 | p-Cymene | 2.6115 | 7.618 | 59456 |
| 11 | Terpinolene | 1.5088 | 8.005 | 34350 |
| 12 | Linalool | 0.0469 | 18.911 | 1067 |
| 13 | Terpinen-4-ol | 67.4166 | 21.667 | 1534859 |
| 14 | a-Terpineol | 1.9131 | 26.479 | 43555 |

As shown in Table 1 above, Blue Curls ‘Pirzada’ essential oil comprises very high levels of terpinen-4-ol, approximately 67.4% (w/v). Tea Tree oil generally has about 35-45% terpinen-4-ol, and thus Blue Curls ‘Pirzada’ essential oil provides a very rich source of terpinen-4-ol.

### EXAMPLE 4

### Isolation of Blue Curls Floral Water

As described in Example 2 above, Blue Curls ‘Pirzada’ floral water is obtained by simple steam distillation of freshly harvested plant material.

After extracting Blue Curls ‘Pirzada’ floral water, it was determined that the floral water has a fresh, earthy aroma and therefore lends itself to a variety of compositions including but not limited to mouthwash, dental floss, lip balm, salve, lotion, toothpaste, deodorant, antiperspirant, bandages, and a host of other cosmetic and health care products.

Using methods readily known in the art, gas chromatography/mass spectrometry revealed that Blue Curls ‘Pirzada’ floral water comprises about 88.5% (w/v) terpinen-4-ol and 2.2% (w/v) terpinen-4-ol acetate.

1. A method for obtaining terpinen-4-ol, comprising
(a) growing a Blue Curls ‘Pirzada’ plant; and
(b) isolating terpinen-4-ol from Blue Curls ‘Pirzada’.

2. The method of claim 1, wherein said terpinen-4-ol is isolated from gland-tipped epidermal trichomes.

3. An essential oil isolated from Blue Curls ‘Pirzada,’ wherein said oil comprises at least about 60% terpinen-4-ol.

4. A composition comprising the essential oil of claim 3.

5. The composition of claim 4, wherein said composition is a pharmaceutical, nutraceutical, cosmetic, lotion, toothpaste, or fragrance.

6. A method for treating a burn in a subject in need thereof comprising:
applying terpinen-4-ol isolated by the method of claim 1 on the burn tissue.

7. A method for treating a microbial tissue infection in a subject in need thereof comprising:
applying terpinen-4-ol isolated by the method of claim 1 on the microbal infected tissue.

8. The method of claim 7, wherein said tissue is skin.

9. (canceled)

10. A method of making the pharmaceutical composition of claim 5 comprising
(a) growing a Blue Curls ‘Pirzada’ plant;
(b) isolating terpinen-4-ol from Blue Curls ‘Pirzada’; and
(c) combining said terpinen-4-ol into with a carrier.

11. A method for increasing terpinen-4-ol content in a plant, comprising crossing a plant with a Blue Curls ‘Pirzada’ plant, wherein Blue Curls ‘Pirzada’ plant is either the pollen or egg donor.

12. (canceled)

13. A floral water isolated from Blue Curls ‘Pirzada,’ wherein said floral water comprises at least about 60% terpinen-4-ol.

14. A composition comprising the floral water of claim 13.
